(19) United States
(12) Patent Application Publication
(10) Pub. No.: US 2021/0338767 A1
(43) Pub. Date: Nov. 4, 2021

(54) METHODS OF SELECTING TREATMENT FOR CXCR4-ASSOCIATED CANCER

(71) Applicants: BioLineRx Ltd., ModiIn (IL); Biokine Therapeutics Ltd., Nes Ziona (IL)

(72) Inventors: Osnat BOHANA-KASHTAN, Tel-Mond (IL); Stephen Michael SHAW, Canterbury, Kent (GB); Amnon PELED, Tel-Aviv (IL)

(73) Assignees: BioLineRx Ltd., ModiIn (IL); Biokine Therapeutics Ltd., Nes Ziona (IL)

(21) Appl. No.: 17/279,775

(22) PCT Filed: Sep. 25, 2019
(86) PCT No.: PCT/IL2019/051057

§ 371 Date: Mar. 25, 2021

Related U.S. Application Data
(60) Provisional application No. 62/735,896, filed on Sep. 25, 2018.

(51) Int. Cl. A61K 38/12; A61K 45/06; A61P 35/02; G01N 33/574

(52) U.S. Cl. CPC A61K 38/12; G01N 33/57426; A61P 35/02; A61K 45/06

(57) ABSTRACT

A method of selecting a treatment regimen for a subject diagnosed with a cancer is provided. The method comprising, determining in cancer cells of said subject, CXCR4 occupancy in a presence and an absence of a peptide having an amino acid sequence as set forth in SEQ ID NO: 1 or an analog or derivative thereof, wherein an increase above a predetermined threshold in said CXCR4 occupancy in said presence of said peptide as compared to said absence of said peptide is indicative of suitability of said subject to treatment with said peptide, or analog or derivative.

### RELATED APPLICATION/S

This application claims priority from U.S. Provisional Patent Application No. 62/735,896 filed on 25 Sep. 2018 which is hereby incorporated by reference in its entirety.

### SEQUENCE LISTING STATEMENT

The ASCII file, entitled 79011 Sequence Listing.txt, created on 23 Sep. 2019, comprising 39,765 bytes, submitted concurrently with the filing of this application is incorporated herein by reference.

### FIELD AND BACKGROUND OF THE INVENTION

The present invention, in some embodiments thereof, relates to methods of selecting treatments and treating CXCR4-associated cancers.

Acute myeloid leukemia is a heterogeneous group of diseases characterized by the uncontrolled proliferation of hematopoietic stem cells and progenitors (blasts) with a reduced capacity to differentiate into mature cells (Estey et al., Lancet 368:1894-1907, 2006). Despite being sensitive to chemotherapeutic agents, long-term disease-free survival for AML patients remains low and the majority eventually relapse from minimal residual disease (MRD; Matsunaga et al., Nat Med. 9:1158-65, 2003).

The bone marrow (BM) is the major site for MRD where adhesion of AML cells to bone marrow components may provide protection from the drugs (Estey et al., Lancet 368:1894-1907, 2006). The chemokine receptor CXCR4 and its ligand stromal derived factor-1 (SDF-1/CXCL12) are involved in the cross-talk between leukemia cells and the BM microenvironment (J. A. Burger and A. Peled, Leukemia 23:43-52, 2009).

The bicyclam drug AMD3100, originally discovered as an anti-HIV compound, specifically interacts with CXCR4 in an antagonistic manner. Blocking CXCR4 receptor with AMD3100 results in the mobilization of hematopoietic progenitor cells. WO 2007/022523 discloses the use of CXCR4 agonists such as AMD3100 for enhancing the effectiveness of chemotherapeutic methods in subjects afflicted with myeloid or hematopoietic malignancies.

T-140 is a 14-residue synthetic peptide developed as a specific CXCR4 antagonist for suppressing HIV-1 (X4-HIV-1) entry to T cells by specifically binding to CXCR4 (Tamamura et al., Biochem. Biophys. Res. Commun. 253(3): 877-882, 1998). Peptide analogs of T-140 were developed as specific CXCR4-antagonisic peptides with inhibitory activity at nanomolar levels [Tamamura et al. (Org. Biomol. Chem. 1: 3663-3669, 2003), WO 2002/020561, WO 2004/020462, WO 2004/087068, WO 00/09152, US 2002/0156034, and WO 2004/024178].

Recently, a comparative study between the CXCR4 antagonists TN140 and AMD3100 suggested that TN140 is more effective than AMD3100 as a monotherapy in AML (Zhang et al., 2012. CXCR4 inhibitors selectively eliminate CXCR4-expressing human acute myeloid leukemia cells in NOG mouse model. Cell Death and Disease 3, e396.). TN140 and to a lesser extent AMD3100 induced regression of human CXCR4-expressing AML cells and targeted the NOD/Shi-scid/IL-2Rγnull (NOG) leukemia-initiating cells (LICs) (Y. Zhang et al., Cell Death and Disease, 2012).

WO 2004/020462 discloses additional novel peptide analogs and derivatives of T-140, including 4F-benzoyl-TN14003.

Beider et al. (Exp. Hematol. 39:282-92, 2011) reported that 4F-benzoyl-TN14003 exhibits a CXCR4-dependent preferential cytotoxicity toward malignant cells of hematopoietic origin including AML and not to normal cells.

WO 2014/155376 discloses the use of 4F-benzoyl-TN14003 combined with a chemotherapeutic agent in the treatment of AML.

WO 2015/063768 discloses the use of 4F-benzoyl-TN14003 in the treatment of AML with FLT3 mutation.

There remains a need for and it would be highly advantageous to have a method of effectively treating AML patients.

### SUMMARY OF THE INVENTION

According to an aspect of some embodiments of the present invention there is provided a method of selecting a treatment regimen for a subject diagnosed with a cancer, the method comprising, determining in cancer cells of the subject, CXCR4 occupancy in a presence and an absence of a peptide having an amino acid sequence as set forth in SEQ ID NO: 1 or an analog or derivative thereof, wherein an increase above a predetermined threshold in the CXCR4 occupancy in the presence of the peptide as compared to the absence of the peptide is indicative of suitability of the subject to treatment with the peptide, or analog or derivative.

According to an aspect of some embodiments of the present invention there is provided a method of treating a cancer in a subject in need thereof, the method comprising:

(a) administering to the subject a therapeutically effective amount of a peptide having an amino acid sequence as set forth in SEQ ID NO: 1 or an analog or derivative thereof; and

(b) determining an increase above a predetermined threshold in CXCR4 occupancy in cancer cells of the subject following the administering, wherein an increase in CXCR4 occupancy following the administering is indicative of an efficacious treatment.

According to an aspect of some embodiments of the present invention there is provided a therapeutically effective amount of a peptide having an amino acid sequence as set forth in SEQ ID NO: 1 or an analog or derivative thereof for use in treating cancer in a subject in need thereof, wherein the therapeutically effective amount is sufficient to induce at least 50% CXCR4 occupancy in cells of the cancer as can be determined by an assay described in Example 2.

According to an aspect of some embodiments of the present invention there is provided a method of treating a cancer in a subject in need thereof, the method comprising administering to the subject a therapeutically effective amount of a peptide having an amino acid sequence as set forth in SEQ ID NO: 1 or an analog or derivative thereof, wherein the therapeutically effective amount is sufficient to induce at least 50% CXCR4 occupancy in cells of the cancer as can be determined by an assay described in Example 2.

According to some embodiments of the invention, the cancer is dependent on CXCR4 for survival.

According to some embodiments of the invention, the cancer is a solid tumor.

According to some embodiments of the invention, the cancer is a hematological malignancy.

According to some embodiments of the invention, the hematological malignancy is acute myeloid leukemia (AML).

According to some embodiments of the invention, the cancer cells of the subject are in a biological sample.

According to some embodiments of the invention, the biological sample is selected from the group consisting of a bone marrow aspirate and a peripheral blood.

According to some embodiments of the invention, the AML is associated with somatic mutation(s).

According to some embodiments of the invention, the somatic mutations are in FLT3. According to some embodiments of the invention, the receptor occupancy is determined using an antibody which binds peptide-free CXCR4 prior to and post contacting with the peptide.

According to some embodiments of the invention, the receptor occupancy is determined a first antibody determining total CXCR4 and a second antibody determining peptide-free CXCR4.

According to some embodiments of the invention, the receptor occupancy is determined by flow cytometry.

According to some embodiments of the invention, the subject diagnosed with AML is in a stage selected from the group consisting of newly diagnosed prior to induction therapy, prior to consolidation therapy, minimal residual disease prior to maintenance therapy, relapsed stage, refractory stage.

According to some embodiments of the invention, the treatment or treating is in combination with another treatment modality.

According to some embodiments of the invention, another treatment modality is selected from the group consisting of a chemotherapy, targeted therapy and an immune modulator.

According to some embodiments of the invention, the immune modulator comprises a checkpoint modulator.

According to some embodiments of the invention, the checkpoint modulator is anti PD-1 or anti PD-L1.

According to some embodiments of the invention, the anti PD-L1 comprises Atezolizumab.

According to some embodiments of the invention, the increase above a predetermined threshold is at least 20%.

According to some embodiments of the invention, the method further comprises treating the subject with the peptide, analog or derivative if the suitability is determined.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows a study design for relapsed/refractory (r/r) AML patients.

FIGS. 2A-D show Median OS and DOR in Subjects Treated with the BL-8040 and HiDAC Combination.

(A) mOS of all subjects enrolled into the study and treated with different BL-8040 dose levels (N=42). (B) mOS of all subjects treated with 1.5 mg/kg BL-8040 (N=23). (C) mOS and (D) DOR in CR and CRi subjects that received 1.5 mg/kg BL-8040 (N=9).

FIGS. 3A-D show BL-8040 Mediated CXCR4 Receptor Occupancy and Induce Mobilization and Differentiation of AML Blasts. (A) Fold change in level of occupied CXCR4 receptor prior and post treatment with low (0.5, 0.75 and 1 mg/kg) and high (1.25, 1.5 and 2 mg/kg) BL-8040 dose levels. (B) Fold change in AML blast counts pre and post BL-8040 and BL-8040+HiDAC treatments in responders and non-responders. (C) Fold change in the level of AML blasts in the BM following BL-8040 treatment (D) Change in levels of BM Granulocytes following 2 days of BL-8040 treatment.

FIG. 4 is a diagram showing current treatment modalities in AML.

### DESCRIPTION OF SPECIFIC EMBODIMENTS OF THE INVENTION

The present invention, in some embodiments thereof, relates to methods of selecting treatments and treating CXCR4-associated cancers.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst conceiving embodiments of the invention, the present inventors aimed at identifying markers that can be used as surrogates or predictors of the clinical efficacy of the peptide set forth in SEQ ID NO: 1 (also referred to as “BL-8040”), derivatives or analogs thereof alone or combined with other treatments.

Thus, according to an aspect of the invention there is provided a method of selecting a treatment regimen for a subject diagnosed with a cancer, the method comprising, determining in cancer cells of said subject, CXCR4 receptor occupancy in a presence and an absence of a peptide having an amino acid sequence as set forth in SEQ ID NO: 1 or an analog or derivative thereof, wherein an increase above a predetermined threshold in said receptor occupancy in said presence of said peptide as compared to said absence of said peptide is indicative of suitability of said subject to treatment with said peptide, or analog or derivative.

The terms “cancer” and “cancerous” refer to or describe the physiological condition in mammals that is typically characterized by unregulated cell growth.

According to a specific embodiment, cells of the cancer is dependent on CXCR4/CXCL12 (SDF-1α, stromal cell-derived factor-1 alpha) for survival.

Accordingly, cells of the cancer express CXCR4. CXCR4 expression can be determined at the mRNA or polypeptide levels, using methods which are well known in the art, e.g., flow cytometry PCR, Western blotting, ELISA, immunohistochemistry and the like.

Cancers which can be treated by the method of this aspect of some embodiments of the invention can be any solid or non-solid cancer (e.g., hematological) and/or cancer metastasis.

According to a specific embodiment, the cancer is a solid tumor.

According another specific embodiment, the cancer is a non-solid tumor.

Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), melanoma cancer, cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; acute myeloblastic leukemia; Multiple Myeloma; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), acute lymphoblastic leukemia (ALL); chronic myeloblastic leukemia (CML); acute myeloblastic leukemia (AML); renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention include metastatic cancers.

According to specific embodiments the cancer is selected from the group consisting of lung cancer, glioma, colon cancer, ovarian cancer, renal cancer, melanoma cancer, hepatocellular cancer, gastric or stomach cancer, glioblastoma, cervical cancer, bladder cancer, breast cancer, colorectal cancer, prostate cancer, thyroid cancer, head and neck and pancreatic cancer.

According to specific embodiments, the cancer is selected from the group consisting of lung cancer, glioma, colon cancer and pancreatic cancer.

According to a specific embodiment, the cancer is a gastric cancer.

According to a specific embodiment, the cancer is a non-small cell lung cancer (NSCLC).

According to yet another embodiment, the cancer is hematological malignancy.

The term “hematological malignancy” herein includes a lymphoma, leukemia, myeloma or a lymphoid malignancy, as well as a cancer of the spleen and the lymph nodes. Exemplary lymphomas that are amenable to treatment with the disclosed agents include both B cell lymphomas and T cell lymphomas. B-cell lymphomas include both Hodgkin's lymphomas and most non-Hodgkins lymphomas. Non-limiting examples of B cell lymphomas include diffuse large B-cell lymphoma (DLBCL), follicular lymphoma (FL), mucosa-associated lymphatic tissue lymphoma (MALT), small cell lymphocytic lymphoma (overlaps with chronic lymphocytic leukemia), mantle cell lymphoma (MCL), Burkitt's lymphoma, mediastinal large B cell lymphoma, Waldenstrom macroglobulinemia, nodal marginal zone B cell lymphoma (NMZL), splenic marginal zone lymphoma (SMZL), intravascular large B-cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma. Hematological malignancies also include leukemia, such as, but not limited to, secondary leukemia, acute myelogenous leukemia (AML; also called acute lymphoid leukemia), chronic myelogenous leukemia (CML), B-cell prolymphocytic leukemia (B-PLL), acute lymphoblastic leukemia (ALL) and myelodysplasia (MDS). Hematological malignancies further include myelomas, such as, but not limited to, multiple myeloma (MM), smoldering multiple myeloma (SMM) and B-cell chronic lymphocytic leukemia (CLL).

According to a particular embodiment, the hematological malignancy is chronic myelogenous leukemia (CML). The term CML includes imatinib-resistant CML, CML tolerant to second/third generation Bcr-Abl TKIs (e.g., dasatinib and nilotinib), imatinib-intolerant CML, accelerated CML, and lymphoid blast phase CML.

Other hematological and/or B cell- or T-cell-associated cancers are encompassed by the term hematological malignancy. For example, hematological malignancies also include cancers of additional hematopoietic cells, including dendritic cells, platelets, erythrocytes, natural killer cells, and polymorphonuclear leukocytes, e.g., basophils, eosinophils, neutrophils and monocytes. It should be clear to those of skill in the art that these pre-malignancies and malignancies will often have different names due to changing systems of classification, and that patients having lymphomas classified under different names may also benefit from the therapeutic regimens of the present invention.

According to specific embodiments, the cancer is selected from the group consisting of multiple myeloma, Lymphoma and leukemia.

According to other specific embodiments, the cancer is selected from the group consisting of multiple myeloma and leukemia.

According to a specific embodiment, the cancer is AML.

According to a specific embodiment, the cancer is T-ALL.

According to a specific embodiment, the cancer is associated with acquired mutations (i.e., somatic mutations).

For instance, in AML, somatic genetic changes are often thought to contribute to leukemogenesis through a “two-hit” process. In other words, for leukemogenesis to occur, two types of mutations, or “two hits,” are typically needed: 1) a mutation that improves hematopoietic cells' ability to proliferate (class I, including FLT3 and KIT), and 2) a mutation that prevents the cells from maturing (class II, including CBFB-MYH11, CEBPA, DEK-NUP214, MLL-MLLT3, NPM1, PML-RARA, RUNX1-RUNX1T1; Naoe and Kiyoi 2013; Shih et al. 2012). Mutations also include epigenetic modifiers such as IDH1, IDH2, and DNMT3A (Naoe and Kiyoi 2013; Shih et al. 2012).

According to a specific embodiment, the mutations are in FLT3.

Genetic variation (e.g., in AML) can be measured using cytogenetics (karyotype and FISH) and/or molecular diagnostics (gene mutations accessed by DNA analysis). Results of these tests are used for patient risk stratification and to guide patient management.

Below, each of the common genetic variants is listed, grouped by risk category in AML (adapted from My Cancer Genome) (Table 1).

TABLE 1
Common Genetic Variants in AML, Grouped by Risk Category.

| | | |
|---|---|---|
| | | Frequency in |
| Cytogenetic Variant | Single Gene Variant(s) | AML |

Favorable Prognosis

| | | |
|---|---|---|
| t(8; 21)^{a, b} | | 7%^{c} |
| t(15; 17)^{a,} _{b} | | 13%^{c} |
| inv(16) or t(16; 16)^{a, b} | | 5%^{c} |
| Normal karyotype | Biallelic CEBPA mutation | 9%e (regardless |
| | positive; FLT3 ITD negative^{a, b, d} | of FLT3 ITD) |
| | NPM1 mutation positive; FLT3 | 26-64%^{e, g} |
| | ITD negative^{b, d, f} | (regardless of |
| | | FLT3 ITD) |
| | NPM1 mutation positive; IDH1 | 3.5%^{h} |
| | or IDH2 mutation positive; FLT3 | |
| | negative^{h} | |

Intermediate Riskⁱ

| | | |
|---|---|---|
| Isolated trisomy 8^{b} | | 10%^{c} |
| t(8; 21)^{b} | KIT mutation | 3%^{j} |
| t(9; 11)^{d} | | 1%^{c} |
| inv(16) or t(16; 16)^{b} | KIT mutation | ~1.5%^{k} |
| Normal karyotype^{a, b} | | 41%^{c} |
| Other cytogenetic | | N/A |
| abnormalities^{c, d} | | |

Poor Risk

| | | |
|---|---|---|
| t(1; 22)^{l} | | <0.5%^{c} |
| inv(3) or 43; 3)^{a, d} | | 1%^{c} |
| Monosomy 5 or 5q-^{a} | | 2%^{c} |
| t(6; 9)^{a, d} | | 1%^{c} |
| Monosomy 7 or 7q-^{a} | | 5%^{c} |
| t(9; 22)^{a, d} | | 1%^{c} |
| 11q23, other than | | 3%^{c} |
| t(9; 11)^{b} | | |
| Normal karyotype | FLT3 ITD^{b, d, h, m} | 27-34%^{h} |
| Monosomal | | 9.3%^{o} |
| karyotype^{n, o} | | |
| Complex karyotype | | 27%^{c} |
| (≥3 clonal | | |
| abnormalities)^{d} | | |

Risk Unclear

| | | |
|---|---|---|
| t(8; 21)^{a, b} | FLT3 mutation or FLT3 ITD^{p} | <1%^{p, q} |
| inv(16) or t(16; 16)^{a, b} | FLT3 mutation or FLT3 ITD^{q} | <1%^{p, q} |
| | CALR mutation | <1%^{s} |
| | DNMT3A mutation^{f, r} | 17%^{e} |
| | IDH1 mutation^{f, r} | 6%^{e} |
| | IDH2 mutation^{f, r} | 9%^{e} |
| | TET2 mutation^{f, r} | 16%^{e} |

AML can be classified according to the FAB or WHO classification systems. Such classifications are provided infra where each of which represents a separate embodiment.

TABLE 2a
WHO classification

| Name | Description |
|---|---|
| Acute myeloid | Includes: |
| leukemia with | AML with translocations between chromosome 8 |
| recurrent genetic | and 21—[t(8; 21)(q22; q22);] RUNX1/RUNX1T1; |
| abnormalities | (ICD-O 9896/3); |
| | AML with inversions in chromosome 16— |
| | [inv(16)(p13.1q22)] or internal translocations in |
| | it—[t(16; 16)(p13.1; q22);] CBFB/MYH11; |
| | (ICD-O 9871/3); |
| | Acute promyelocytic leukemia with translocations |
| | between chromosome 15 and 17—[t(15; 17)(q22; |
| | q12);] RARA/PML; (ICD-O 9866/3); |
| | AML with translocations between chromosome 9 |
| | and 11—[t(9; 11)(p22; q23);] MLLT3/MLL; |
| | AML with translocations between chromosome 6 |
| | and 9—[t(6; 9)(p23; q34);] DEK/NUP214; |
| | AML with inversions in chromosome 3— |
| | [inv(3)(q21q26.2)] or internal translocations in it— |
| | [t(3; 3)(q21; q26.2);] RPN1/EVI1; |
| | Megakaryoblastic AML with translocations |
| | between chromosome 1 and 22—[t(1; 22)(p13; |
| | q13);] RBM15/MKL1; |
| | AML with mutated NPM1 |
| | AML with mutated CEBPA |
| AML with | This category includes people who have had a |
| myelodysplasia- | prior documented myelodysplastic syndrome |
| related changes | (MDS) or myeloproliferative disease (MPD) that |
| | then has transformed into AML, or who have |
| | cytogenetic abnormalities characteristic for this |
| | type of AML (with previous history of MDS or |
| | MPD that has gone unnoticed in the past, but the |
| | cytogenetics is still suggestive of MDS/MPD |
| | history). This elderly people and often has a |
| | worse prognosis. Includes: |
| | AML with complex karyotype |
| | Unbalanced abnormalities |
| | AML with deletions of chromosome 7—[del(7q);] |
| | AML with deletions of chromosome 5—[del(5q);] |
| | AML with unbalanced chromosomal aberrations in |
| | chromosome 17—[i(17q)/t(17p);] |
| | AML with deletions of chromosome 13— |
| | [del(13q);] |
| | AML with deletions of chromosome 11— |
| | [del(11q);] |
| | AML with unbalanced chromosomal aberrations in |
| | chromosome 12—[del(12p)/t(12p);] |
| | AML with deletions of chromosome 9—[del(9q);] |
| | AML with aberrations in chromosome X— |
| | [idic(X)(q13);] |
| | Balanced abnormalities |
| | AML with translocations between chromosome 11 |
| | and 16—[t(11; 16)(q23; q13.3);], unrelated to |
| | previous chemotherapy or ionizing radiation |
| | AML with translocations between chromosome 3 |
| | and 21—[t(3; 21)(q26.2; q22.1);], unrelated to |
| | previous chemotherapy or ionizing radiation |
| | AML with translocations between chromosome 1 |
| | and 3—[t(1; 3)(p36.3; q21.1);] |
| | AML with translocations between chromosome 2 |
| | and 11—[t(2; 11)(p21; q23);], unrelated to |
| | previous chemotherapy or ionizing radiation |
| | AML with translocations between chromosome 5 |
| | and 12—[t(5; 12)(q33; p12);] |
| | AML with translocations between chromosome 5 |
| | and 7—[t(5; 7)(q33; q11.2);] |
| | AML with translocations between chromosome 5 |
| | and 17—[t(5; 17)(q33; p13);] |
| | AML with translocations between chromosome 5 |
| | and 10—[t(5; 10)(q33; q21);] |
| | AML with translocations between chromosome 3 |
| | and 5—[t(3; 5)(q25; q34);] |
| Therapy-related | This category includes people who have had prior |
| myeloid neoplasms | chemotherapy and/or radiation and subsequently |
| | develop AML or MDS. These leukemias may be |
| | characterized by specific chromosomal |
| | abnormalities, and often carry a worse prognosis. |
| Myeloid sarcoma | This category includes myeloid sarcoma. |
| Myeloid | This category includes so-called “transient |
| proliferations related | abnormal myelopoiesis” and “Myeloid leukemia |
| to Down syndrome | associated with Down syndrome” |
| Blastic plasmacytoid | This category includes so-called “blastic |
| dendritic cell | plasmacytoid dendritic cell neoplasm” |
| neoplasm | |
| AML not otherwise | Includes subtypes of AML that do not fall into the |
| categorized | above categories |
| | AML with minimal differentiation |
| | AML without maturation |
| | AML with maturation |
| | Acute myelomonocytic leukemia |
| | Acute monoblastic and monocytic leukemia |
| | Acute erythroid leukemia |
| | Acute megakaryoblastic leukemia |
| | Acute basophilic leukemia |
| | Acute panmyelosis with myelofibrosis |

TABLE 2b
FAB subtypes

| Type | Name | Cytogenetics |
|---|---|---|
| M0 | acute blast cellic leukemia, minimally | |
| | differentiated | |
| M1 | acute blast cellic leukemia, without | |
| | maturation | |
| M2 | acute blast cellic leukemia, with | t(8; 21)(q22; |
| | granulocytic maturation | q22), t(6; 9) |
| M3 | promyelocytic, or acute promyelocytic | t(15; 17) |
| | leukemia (APL) | |
| M4 | acute myelomonocytic leukemia | inv(16)(p13q22), |
| | | del(16q) |
| M4eo | myelomonocytic together with bone | inv(16), t(16; 16) |
| | marrow eosinophilia | |
| M5 | acute monoblastic leukemia (M5a) or | del (11q), |
| | acute monocytic leukemia (M5b) | t(9; 11), t(11; 19) |
| M6 | acute erythroid leukemias, including | |
| | erythroleukemia (M6a) and very rare | |
| | pure erythroid leukemia (M6b) | |
| M7 | acute megakaryoblastic leukemia | t(1; 22) |

According to a specific embodiment the disease is characterized by a mutation in a FLT3 gene.

Internal tandem duplication in FLT3 gene is typically characterized by aberrant RNA transcripts which may stem from a simple internal duplication within exon 11; internal duplication (26 bp) with a 4-bp insertion; or a 136-bp sequence from the 3′ part of exon 11 to intron 11 and the first 16-bp sequence of exon 12 are duplicated with 1-bp insertion. Other abnormalities may also exist.

According to a specific embodiment, the FLT3 mutation results in activation of the protein.

In one embodiment the FLT3 mutation is a FLT3 internal-tandem duplication (ITD) mutation (Levis and Small, Leukemia 17: 1738-1752, 2003).

According to another embodiment the FLT3 mutation is a missense mutation at aspartic acid residue 835.

As used herein, the term “subject” includes mammals, preferably human beings at any age diagnosed with cancer.

According to a specific embodiment, the subject is at a stage selected from the group consisting of relapsed and/or refractory (r/r), prior to or following induction, prior to or following consolidation, and prior to maintenance and minimal residual disease (MRD). Those stages are well known to those of skills in the art of oncology.

According to a specific embodiment, the method is effected ex-vivo, whereby the peptide is contacted with cancer cells ex-vivo, though other modes of detection are also contemplated. For instance, in vivo contacting with the peptide followed by occupancy assessment e.g., ex-vivo.

As used herein cells of the subject, refers to tumor cells such as comprised in a biological sample.

Cells can be from the peripheral blood.

Cells can be from the bone marrow (e.g., by bone marrow aspiration).

Cells can be from the tumor in the case of a solid tumor (e.g., biopsy).

Such biological samples include, but are not limited to, tissues, cells and body fluids such as whole blood, serum, plasma, cerebrospinal fluid, urine, lymph fluids, and various external secretions of the respiratory, intestinal and genitourinary tracts, tears, saliva, milk as well as white blood cells, malignant tissues, amniotic fluid and ascites fluid.

As used herein “receptor occupancy” refers to CXCR4 receptor occupancy.

Occupancy refers to occupancy with a CXCR4 binding agent e.g., CXCR4 binding drug e.g., the peptide as described herein.

The CXCR4 binding agent can be a natural ligand e.g., CXCL12.

The CXCR4 binding agent can be a CXCR4 binding drug, such as a CXCR4 inhibitor, antagonist, super-agonist etc.

According to a specific embodiment, the CXCR4 binding drug is a peptide.

According to a specific embodiment, the receptor occupancy assay detects only the binding of the CXCR binding drug (or absence of a drug-occupied CXCR4) and not binding of natural ligand-bound CXCR4 (CXCL12).

As used herein, the term “peptide” encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells.

According to a specific embodiment, the peptide is 5-100 amino acids in length.

According to a specific embodiment, the peptide is 5-50 amino acids in length. According to a specific embodiment, the peptide is 5-20 amino acids in length. According to a specific embodiment, the peptide is 5-15 amino acids in length. According to a specific embodiment, the peptide is 10-20 amino acids in length. According to a specific embodiment, the peptide is 10-15 amino acids in length.

According to specific embodiments, the CXCR4-antagonistic peptides of the present invention are for example, 4F-benzoyl-TN14003 (SEQ ID NO: 1) analogs and derivatives and are structurally and functionally related to the peptides disclosed in patent applications WO 2002/020561 and WO 2004/020462, also known as “T-140 analogs”, as detailed hereinbelow.

In various particular embodiments, the T-140 analog or derivative has an amino acid sequence as set forth in the following formula (I) or a salt thereof:

(I)
1 2 3 4 5 6 7 8 9 10 11 12 13 14
A₁-A₂-A₃-Cys-Tyr-A₄-A₅-A₆-A₇-A₈-A₉-A₁₀-Cys-A₁₁

wherein:

A₁ is an arginine, lysine, ornithine, citrulline, alanine or glutamic acid residue or a N-α-substituted derivative of these amino acids, or Ai is absent;

A₂ represents an arginine or glutamic acid residue if A₁ is present, or A₂ represents an arginine or glutamic acid residue or a N-α-substituted derivative of these amino acids if A₁ is absent;

A₃ represents an aromatic amino acid residue;

A₄, A₅ and A₉ each independently represents an arginine, lysine, ornithine, citrulline, alanine or glutamic acid residue;

A₆ represents a proline, glycine, ornithine, lysine, alanine, citrulline, arginine or glutamic acid residue;

A₇ represents a proline, glycine, ornithine, lysine, alanine, citrulline or arginine residue;

A₈ represents a tyrosine, phenylalanine, alanine, naphthylalanine, citrulline or glutamic acid residue;

A₁₀ represents a citrulline, glutamic acid, arginine or lysine residue;

A₁₁ represents an arginine, glutamic acid, lysine or citrulline residue wherein the C-terminal carboxyl may be derivatized;

and the cysteine residue of the 4-position or the 13-position can form a disulfide bond, and the amino acids can be of either L or D form.

Exemplary peptides according to formula (I) are peptides having an amino acid sequence as set forth in any one of SEQ ID NOS:1-72, as presented in Table 3 hereinbelow.

TABLE 3
T-140 and currently preferred T-140 analogs

| | SEQ ID | |
|---|---|---|
| Analog | NO: | Amino acid sequence |
| 4F-benzoyl- | 1 | 4F-benzoyl-Arg-Arg-Nal-Cys-Tyr-Cit-Lys-DLys-Pro-Tyr-Arg-Cit-Cys- |
| TN14003 | | Arg-NH₂ |
| AcTC14003 | 2 | Ac-Arg-Arg-Nal-Cys-Tyr-Cit-Lys-DLys-Pro-Tyr-Arg-Cit-Cys-Arg-OH |
| AcTC14005 | 3 | Ac-Arg-Arg-Nal-Cys-Tyr-Arg-Lys-DCit-Pro-Tyr-Arg-Cit-Cys-Arg-OH |
| AcTC14011 | 4 | Ac-Arg-Arg-Nal-Cys-Tyr-Cit-Lys-DCit-Pro-Tyr-Arg-Cit-Cys-Arg-OH |
| AcTC14013 | 5 | Ac-Arg-Arg-Nal-Cys-Tyr-Cit-Lys-DLys-Pro-Tyr-Cit-Cit-Cys-Arg-OH |
| AcTC14015 | 6 | Ac-Cit-Arg-Nal-Cys-Tyr-Cit-Lys-DLys-Pro-Tyr-Arg-Cit-Cys-Arg-OH |
| AcTC14017 | 7 | Ac-Cit-Arg-Nal-Cys-Tyr-Arg-Lys-DCit-Pro-Tyr-Arg-Cit-Cys-Arg-OH |
| AcTC14019 | 8 | Ac-Arg-Arg-Nal-Cys-Tyr-Arg-Lys-DCit-Pro-Tyr-Cit-Cit-Cys-Arg-OH |
| AcTC14021 | 9 | Ac-Cit-Arg-Nal-Cys-Tyr-Arg-Lys-DLys-Pro-Tyr-Cit-Cit-Cys-Arg-OH |
| AcTC14012 | 10 | Ac-Arg-Arg-Nal-Cys-Tyr-Cit-Lys-DCit-Pro-Tyr-Arg-Cit-Cys-Arg-NH₂ |
| AcTC14014 | 11 | Ac-Arg-Arg-Nal-Cys-Tyr-Cit-Lys-DLys-Pro-Tyr-Cit-Cit-Cys-Arg-NH₂ |
| AcTC14016 | 12 | Ac-Cit-Arg-Nal-Cys-Tyr-Cit-Lys-DLys-Pro-Tyr-Arg-Cit-Cys-Arg-NH₂ |
| AcTC14018 | 13 | Ac-Cit-Arg-Nal-Cys-Tyr-Arg-Lys-DCit-Pro-Tyr-Arg-Cit-Cys-Arg-NH₂ |
| AcTC14020 | 14 | Ac-Arg-Arg-Nal-Cys-Tyr-Arg-Lys-DCit-Pro-Tyr-Cit-Cit-Cys-Arg-NH₂ |
| AcTC14022 | 15 | Ac-Cit-Arg-Nal-Cys-Tyr-Arg-Lys-DLys-Pro-Tyr-Cit-Cit-Cys-Arg-NH₂ |
| TE14001 | 16 | H-DGlu-Arg-Nal-Cys-Tyr-Arg-Lys-DLys-Pro-Tyr-Arg-Cit-Cys-Arg-OH |
| TE14002 | 17 | H-Arg-Glu-Nal-Cys-Tyr-Arg-Lys-DLys-Pro-Tyr-Arg-Cit-Cys-Arg-OH |
| TE14003 | 18 | H-Arg-Arg-Nal-Cys-Tyr-Glu-Lys-DLys-Pro-Tyr-Arg-Cit-Cys-Arg-OH |
| TE14004 | 19 | H-Arg-Arg-Nal-Cys-Tyr-Arg-Glu-DLys-Pro-Tyr-Arg-Cit-Cys-Arg-OH |
| TE14005 | 20 | H-Arg-Arg-Nal-Cys-Tyr-Arg-Lys-DGlu-Pro-Tyr-Arg-Cit-Cys-Arg-OH |
| TE14006 | 21 | H-Arg-Arg-Nal-Cys-Tyr-Arg-Lys-DLys-Pro-Tyr-Glu-Cit-Cys-Arg-OH |
| TE14007 | 22 | H-Arg-Arg-Nal-Cys-Tyr-Arg-Lys-DLys-Pro-Tyr-Arg-Cit-Cys-Glu-OH |
| TE14011 | 23 | H-Arg-Arg-Nal-Cys-Tyr-Cit-Lys-DGlu-Pro-Tyr-Arg-Cit-Cys-Arg-NH₂ |
| TE14012 | 24 | H-Arg-Arg-Nal-Cys-Tyr-DGlu-Lys-DCit-Pro-Tyr-Arg-Cit-Cys-Arg-NH₂ |
| TE14013 | 25 | H-Arg-Arg-Nal-Cys-Tyr-DGlu-Lys-DGlu-Pro-Tyr-Arg-Cit-Cys-Arg-NH₂ |
| TE14014 | 26 | H-DGlu-Arg-Nal-Cys-Tyr-Cit-Lys-DGlu-Pro-Tyr-Arg-Cit-Cys-Arg-NH₂ |
| TE14015 | 27 | H-Arg-Arg-Nal-Cys-Tyr-Cit-Lys-DGlu-Pro-DGlu-Arg-Cit-Cys-Arg-NH₂ |
| TE14016 | 28 | H-Arg-Arg-Nal-Cys-Tyr-Cit-Lys-DGlu-Pro-Tyr-Arg-DGlu-Cys-Arg-NH2 |
| AcTE14014 | 29 | Ac-DGlu-Arg-Nal-Cys-Tyr-Cit-Lys-DGlu-Pro-Tyr-Arg-Cit-Cys-Arg-NH₂ |
| AcTE14015 | 30 | Ac-Arg-Arg-Nal-Cys-Tyr-Cit-Lys-DGlu-Pro-DGlu-Arg-Cit-Cys-Arg-NH₂ |
| AcTE14016 | 31 | Ac-Arg-Arg-Nal-Cys-Tyr-Cit-Lys-DGlu-Pro-Tyr-Arg-DGlu-Cys-Arg-NH₂ |
| TF1: | 32 | Ac-Arg-Arg-Nal-Cys-Tyr-Cit-Lys-DGlu-Pro-Tyr-Arg-Cit-Cys-Arg-NH₂ |
| AcTE14011 | | |
| TF2: guanyl- | 33 | guanyl-Arg-Arg-Nal-Cys-Tyr-Cit-Lys-DGlu-Pro-Tyr-Arg-Cit-Cys- |
| TE14011 | | Arg-NH₂ |
| TF3: | 34 | TMguanyl-Arg-Arg-Nal-Cys-Tyr-Cit-Lys-DGlu-Pro-Tyr-Arg-Cit-Cys- |
| TMguanyl- | | Arg-NH₂ |
| TE14011 | | |
| TF4: | 35 | TMguanyl-Arg-Nal-Cys-Tyr-Cit-Lys-DGlu-Pro-Tyr-Arg-Cit-Cys- |
| TMguanyl- | | Arg-NH₂ |
| TE14011 (2-14) | | |
| TF5: 4F- | 36 | 4F-benzoyl-Arg-Arg-Nal-Cys-Tyr-Cit-Lys-DGlu-Pro-Tyr-Arg-Cit-Cys- |
| benzoyl- | | Arg-NH₂ |
| TE14011 | | |
| TF6: 2F- | 37 | 2F-benzoyl-Arg-Arg-Nal-Cys-Tyr-Cit-Lys-DGlu-Pro-Tyr-Arg-Cit-Cys- |
| benzoyl- | | Arg-NH₂ |
| TE14011 | | |
| TF7: APA- | 38 | APA-Arg-Nal-Cys-Tyr-Cit-Lys-DGlu-Pro-Tyr-Arg-Cit-Cys-Arg-NH₂ |
| TE14011 (2-14) | | |
| TF8: desamino- | 39 | desamino-R-Arg-Nal-Cys-Tyr-Cit-Lys-DGlu-Pro-Tyr-Arg-Cit-Cys- |
| R-TE14011 (2- | | Arg-NH₂ |
| 14) | | |
| TF9: guanyl- | 40 | Guanyl-Arg-Nal-Cys-Tyr-Cit-Lys-DGlu-Pro-Tyr-Arg-Cit-Cys-Arg-NH₂ |
| TE14011 (2-14) | | |
| TF10: succinyl- | 41 | succinyl-Arg-Nal-Cys-Tyr-Cit-Lys-DGlu-Pro-Tyr-Arg-Cit-Cys-Arg-NH₂ |
| TE14011 (2-14) | | |
| TF11: glutaryl- | 42 | glutaryl-Arg-Nal-Cys-Tyr-Cit-Lys-DGlu-Pro-Tyr-Arg-Cit-Cys-Arg-NH₂ |
| TE14011 (2-14) | | |
| TF12: | 43 | deaminoTMG-APA-Arg-Nal-Cys-Tyr-Cit-Lys-DGlu-Pro-Tyr-Arg-Cit-Cys- |
| deaminoTMG- | | Arg-NH₂ |
| APA-TE14011 | | |
| (2-14) | | |
| TF15: H-Arg- | 44 | R-CH2-Arg-Nal-Cys-Tyr-Cit-Lys-DGlu-Pro-Tyr-Arg-Cit-Cys-Arg-NH₂ |
| CH2NH- | | |
| RTE14011 (2- | | |
| 14) | | |
| TF17: TE14011 | 45 | H-Arg-Nal-Cys-Tyr-Cit-Lys-DGlu-Pro-Tyr-Arg-Cit-Cys-Arg-NH₂ |
| (2-14) | | |
| TF18: | 46 | TMguanyl-Arg-Arg-Nal-Cys-Tyr-Cit-Lys-DCit-Pro-Tyr-Arg-Cit-Cys- |
| TMguanyl- | | Arg-NH₂ |
| TC14012 | | |
| TF19: ACA- | 47 | ACA-Arg-Arg-Nal-Cys-Tyr-Cit-Lys-DCit-Pro-Tyr-Arg-Cit-Cys-Arg-NH₂ |
| TC14012 | | |
| TF20: ACA- | 48 | ACA-Arg-Arg-Nal-Cys-Tyr-Arg-Lys-DLys-Pro-Tyr-Arg-Cit-Cys-Arg-OH |
| T140 | | |
| TZ14011 | 49 | H-Arg-Arg-Nal-Cys-Tyr-Cit-Arg-DLys-Pro-Tyr-Arg-Cit-Cys-Arg-NH₂ |
| AcTZ14011 | 50 | Ac-Arg-Arg-Nal-Cys-Tyr-Cit-Arg-DLys-Pro-Tyr-Arg-Cit-Cys-Arg-NH₂ |
| AcTN14003 | 51 | Ac-Arg-Arg-Nal-Cys-Tyr-Cit-Lys-DLys-Pro-Tyr-Arg-Cit-Cys-Arg-NH₂ |
| AcTN14005 | 52 | Ac-Arg-Arg-Nal-Cys-Tyr-Arg-Lys-DCit-Pro-Tyr-Arg-Cit-Cys-Arg-NH₂ |
| 4F-benzoyl- | 53 | 4F-benzoyl-Arg-Arg-Nal-Cys-Tyr-Cit-Lys-DGlu-Pro-Tyr-Arg-Cit-Cys- |
| TN14011-Me | | Arg-NHMe |
| 4F-benzoyl- | 54 | 4F-benzoyl-Arg-Arg-Nal-Cys-Tyr-Cit-Lys-DGlu-Pro-Tyr-Arg-Cit-Cys- |
| TN14011-Et | | Arg-NHEt |
| 4F-benzoyl- | 55 | 4F-benzoyl-Arg-Arg-Nal-Cys-Tyr-Cit-Lys-DGlu-Pro-Tyr-Arg-Cit-Cys- |
| TN14011-iPr | | Arg-NHiPr |
| 4F-benzoyl- | 56 | 4F-benzoyl-Arg-Arg-Nal-Cys-Tyr-Cit-Lys-DGlu-Pro-Tyr-Arg-Cit-Cys- |
| TN14011- | | Arg-tyramine |
| tyramine | | |
| TA14001 | 57 | H-Ala-Arg-Nal-Cys-Tyr-Arg-Lys-DLys-Pro-Tyr-Arg-Cit-Cys-Arg-OH |
| TA14005 | 58 | H-Arg-Arg-Nal-Cys-Tyr-Ala-Lys-DLys-Pro-Tyr-Arg-Cit-Cys-Arg-OH |
| TA14006 | 59 | H-Arg-Arg-Nal-Cys-Tyr-Arg-Ala-DLys-Pro-Tyr-Arg-Cit-Cys-Arg-OH |
| TA14007 | 60 | H-Arg-Arg-Nal-Cys-Tyr-Arg-Lys-DAla-Pro-Tyr-Arg-Cit-Cys-Arg-OH |
| TA14008 | 61 | H-Arg-Arg-Nal-Cys-Tyr-Arg-Lys-DLys-Ala-Tyr-Arg-Cit-Cys-Arg-OH |
| TA14009 | 62 | H-Arg-Arg-Nal-Cys-Tyr-Arg-Lys-DLys-Pro-Ala-Arg-Cit-Cys-Arg-OH |
| TA14010 | 63 | H-Arg-Arg-Nal-Cys-Tyr-Arg-Lys-DLys-Pro-Tyr-Ala-Cit-Cys-Arg-OH |
| TC14001 | 64 | H-Cit-Arg-Nal-Cys-Tyr-Arg-Lys-DLys-Pro-Tyr-Arg-Cit-Cys-Arg-OH |
| TC14003 | 65 | H-Arg-Arg-Nal-Cys-Tyr-Cit-Lys-DLys-Pro-Tyr-Arg-Cit-Cys-Arg-OH |
| TN14003 | 66 | H-Arg-Arg-Nal-Cys-Tyr-Cit-Lys-DLys-Pro-Tyr-Arg-Cit-Cys-Arg-NH₂ |
| TC14004 | 67 | H-Arg-Arg-Nal-Cys-Tyr-Arg-Cit-DLys-Pro-Tyr-Arg-Cit-Cys-Arg-OH |
| TC14012 | 68 | H-Arg-Arg-Nal-Cys-Tyr-Cit-Lys-DCit-Pro-Tyr-Arg-Cit-Cys-Arg-NH₂ |
| T-140 | 69 | H-Arg-Arg-Nal-Cys-Tyr-Arg-Lys-DLys-Pro-Tyr-Arg-Cit-Cys-Arg-OH |
| TC14011 | 70 | H-Arg-Arg-Nal-Cys-Tyr-Cit-Lys-DCit-Pro-Tyr-Arg-Cit-Cys-Arg-OH |
| TC14005 | 71 | H-Arg-Arg-Nal-Cys-Tyr-Arg-Lys-DCit-Pro-Tyr-Arg-Cit-Cys-Arg-OH |
| TC14018 | 72 | H-Cit-Arg-Nal-Cys-Tyr-Arg-Lys-DCit-Pro-Tyr-Arg-Cit-Cys-Arg-NH₂ |

According to a specific embodiment, in each one of SEQ ID NOS:1-72, two cysteine residues are coupled in a disulfide bond.

In another embodiment, the analog or derivative has an amino acid sequence as set forth in SEQ ID NO:65 (H-Arg-Arg-Nal-Cys-Tyr-Cit-Lys-DLys-Pro-Tyr-Arg-Cit-Cys-Arg-OH; TC14003).

In another embodiment, the peptide used in the compositions and methods of the invention consists essentially of an amino acid sequence as set forth in SEQ ID NO:1. In another embodiment, the peptide used in the compositions and methods of the invention comprises an amino acid sequence as set forth in SEQ ID NO:1. In another embodiment, the peptide is at least 60%, at least 70% or at least 80% homologous to SEQ ID NO:1. In another embodiment, the peptide is at least 90% homologous to SEQ ID NO:1. In another embodiment, the peptide is at least about 95% homologous to SEQ ID NO:1. Each possibility represents a separate embodiment of the present invention.

In various other embodiments, the peptide is selected from SEQ ID NOS:1-72, wherein each possibility represents a separate embodiment of the present invention.

In another embodiment, the peptide has an amino acid sequence as set forth in any one of SEQ ID NOS: 1-4, 10, 46, 47, 51-56, 65, 66, 68, 70 and 71. In another embodiment, the peptide has an amino acid sequence as set forth in any one of SEQ ID NOS: 4, 10, 46, 47, 68 and 70. In another embodiment, the peptide has an amino acid sequence as set forth in any one of SEQ ID NOS:1, 2, 51, 65 and 66. In another embodiment, the peptide has an amino acid sequence as set forth in any one of SEQ ID NOS:53-56.

In an embodiment, the peptide has an amino acid sequence as set forth in SEQ ID NO:1. In another embodiment, the peptide has an amino acid sequence as set forth in SEQ ID NO:2. In another embodiment, the peptide has an amino acid sequence as set forth in SEQ ID NO:51. In another embodiment, the peptide has an amino acid sequence as set forth in SEQ ID NO:66.

According to a preferred embodiment, the CXCR4 antagonist is as set forth in SEQ ID NO: 1, also termed BL-8040 and BKT140.

Identical aliquots of the cells of a sample (e.g., BM and/or PB) are contacted with the peptide (at a single or varying concentrations, to determine dose dependency and also to evaluate the effective dose) or treated with control (e.g., buffer and optionally irrelevant peptide e.g., scrambled peptide not able to bind CXCR4), the first being referred to as “presence of the peptide”, while the latter being referred to as “absence of the peptide”.

Methods of determining receptor occupancy are well known in the art.

Receptor occupancy is typically assessed using two monoclonal antibodies (mAbs) binding to two different epitopes of the CXCR4 antigen. A first antibody is specific for the same epitope binding the peptide, as described above, e.g., 12G5 (Abraham et al. Clin Cancer Res. 2017 Nov. 15;23(22):6790-6801. doi: 10.1158/1078-0432.CCR-16-2919. Epub 2017 Aug. 23), and thus a reporter of the free CXCR4 sites (unoccupied by e.g. the peptide e.g., SEQ ID NO: 1). The second antibody, e.g., 1D9 (Abraham et al. supra), directed to a different epitope than that of the first antibody, provides a positive control for the presence of CXCR4+ cells in addition to the result seen with 12G5 prior to treatment with BL-8040. It will be appreciated that receptor occupancy can also be determined by using an antibody which binds peptide-free CXCR4 prior to and post contacting with the peptide (e.g., 12G5, supra).

The use of beads as calibrators and an indirect detection allows a quantitative approach without any modification of binding capacity of the peptide. The combination of the results allows quantification of CXCR4 occupied cells.

The detection can be done in parallel with other markers for the disease. For instance in the case of AML, the following markers can be used AML panel (CD45, CD34, CD33, CD117, HLA-DR), AML-MRD panels (Panel 1: CD13, CD15, CD19, CD33, CD34, CD38, CD45, CD71, CD117, HLA-DR; Panel 2: CD4, CD13, CD14, CD16, CD34, CD38, CD45, CD64, CD123, HLA-DR; Panel 3: CD5, CD7, CD11b, CD33, CD34, CD38, CD45, CD56), etc. To any of which a CXCR4 antibody can be joined.

Determination of receptor occupancy is typically performed by flow cytometry, since it allows cell based assessments.

Receptor occupancy can also be determined by other means. These include, the use of radioactive isotopes, luminescence e.g., horseradish peroxidase and the like.

According to a specific embodiment, the receptor occupancy is determined as follows: Percent or number of CXCR4 expressing cells (e.g., using 12G5 Ab) post drug (e.g., SEQ ID NO: 1) treatment out of percent or number of CXCR4 expressing cells (e.g., using 12G5 Ab) prior to drug (e.g., SEQ ID NO: 1) treatment *100, such a calculation provides receptor occupancy per cell.

For example, tumor cells (e.g., AML blasts) from patients are isolated at screening e.g., from peripheral blood and/or bone marrow. The cells are then treated with various dose levels of the peptide or with control and stained for CXCR4 e.g., using clones 1D9 (which is able to bind CXCR4 even when the receptor is already bound by BL-8040 and allows determination of total CXCR4 expression) and 12G5 (which is unable to bind CXCR4 when the receptor is already bound by BL-8040 and allows determination of receptor occupancy) as well as for the EuroFlow AML panel (CD45, CD34, CD33, CD117, HLA-DR) or the AML MRD panels (Panel 1: CD13, CD15, CD19, CD33, CD34, CD38, CD45, CD71, CD117, HLA-DR; Panel 2: CD4, CD13, CD14, CD16, CD34, CD38, CD45, CD64, CD123, HLA-DR; Panel 3: CD5, CD7, CD11b, CD33, CD34, CD38, CD45, CD56) and analyzed by flow cytometry.

According to a specific embodiment, the antibodies are fluorescently labeled with fluorophores. For example, 1D9 can be labeled with PE and 12G5 can be labeled with APC. The selection of the fluorophore will depend on other fluorophores used in the assay, so that a distinctive signal is obtained.

Beads (such as Trucount tubes BD Cat #340334) can be used to measure absolute number of cells that express CXCR4 and number of cells that have occupied CXCR4

Patients with cancer (e.g., AML) cells that express CXCR4 and demonstrate an increase in CXCR4 receptor occupancy of at least 20%, 30%, 40%, 50% (e.g., at least 60%, at least 70%, at least 80%, at least 90%, at least 100%, at least 200%, at least 300%, at least 400%, at least 500%, at least 600%, at least 700%, at least 800%, at least 900%, at least 1000%) e.g., by flow cytometry are selected for peptide treatment.

Additional tests to measure CXCR4 expression can be based on assessment of protein expression (by ELISA or other quantitative method), or mRNA level (by RT-QPCR).

A subject with upregulated CXCR4 is also characterized for mutations (e.g., in AML mutations in FLT3 for instance) to enable identification of additional genetic biomarkers to stratify patients. The Illumina TruSight myeloid panel (or other accepted panels) that consists of genes selected by panels of experts in the areas of myeloid hematological cancers to cover key mutations found in AML, can be used (some are also listed above).

According to a specific embodiment, the method further comprises treating the subject with the peptide, analog or derivative if found suitable for treatment, as described above.

The terms “treatment” or “treating” as used herein interchangeably refer to arresting the development of a pathology (disease, disorder or condition i.e., acute myeloid leukemia) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

The present teachings can also be used to assess treatment efficacy, also referred to as “monitoring treatment”.

Accordingly, there is provided a method of treating a cancer in a subject in need thereof, the method comprising:

(a) administering to the subject a therapeutically effective amount of a peptide having an amino acid sequence as set forth in SEQ ID NO: 1 or an analog or derivative thereof; and
(b) determining an increase in CXCR4 occupancy in cancer cells of the subject following said administering, wherein an increase in CXCR4 occupancy following said administering is indicative of an efficacious treatment.

According to a specific embodiment, said determining is effected between two or more administrations of the peptide (e.g., in time intervals).

According to a specific embodiment, an increase above a statistically significant threshold e.g., at least 20%, at least 30%, at least 40%, at least 50%, at least 55%, at least 60%, at least 65%, at least 70%, at least 75%, at least 80%, at least 85%, at least 90%, at least 95%, at least 100%, is indicative of an efficacious treatment, wherein the increase is determined as compared to prior to a previous administration of the peptide or prior to a first administration of the peptide.

Accordingly, if response is inadequate as determined by any parameter known in the art e.g., CR, Cri, PR, overall survival, disease-free survival, stable disease; and optionally the threshold is not met then the treatment with the peptide is terminated. Conversely, if an increase, as defined above, is determined, the physician (e.g., oncologist) may advise repeating treatment with the peptide.

The receptor occupancy assay, as described herein, may be used to personalize the peptide dose, ultimately reducing the amount and/or number of administrations.

Thus, according to an aspect of the invention there is provided a method of treating a cancer (e.g., AML) in a subject in need thereof, the method comprising administering to the subject a therapeutically effective amount of a peptide having an amino acid sequence as set forth in SEQ ID NO: 1 or an analog or derivative thereof, wherein said therapeutically effective amount is sufficient to induce at least 50% CXCR4 occupancy in cells of the cancer (e.g., as determined in bone marrow aspirates and/or peripheral blood) as can be determined by an assay described herein e.g., Example 2.

Any of the treatment modalities described herein can be combined with other treatments which can alleviate cancer (also referred to herein as “an anti-cancer agent”).

According to specific embodiments, the peptide can be administered to a subject in combination with other established or experimental therapeutic regimen to treat cancer including analgetics, chemotherapeutic agents, radiotherapeutic agents, hormonal therapy, immune modulators, engineered immune cell therapy (e.g., CAR-T) and other treatment regimens (e.g., surgery, cell transplantation e.g. hematopoietic stem cell transplantation) which are well known in the art.

The chemotherapeutic agent of the present invention can be, but not limited to, cytarabine (cytosine arabinoside, Ara-C, Cytosar-U), asprin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2⋅difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including Nmethylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

In some embodiments the chemotherapeutic agent of the present invention is cytarabine (cytosine arabinoside, Ara-C, Cytosar-U), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, Cytosar-U), melphalan, busulfan (Myleran®), thiotepa, bleomycin, platinum (cisplatin), cyclophosphamide, Cytoxan®), daunorubicin, doxorubicin, idarubicin, mitoxantrone, 5-azacytidine, cladribine, fludarabine, hydroxyurea, 6-mercaptopurine, methotrexate, 6-thioguanine, or any combination thereof.

In an embodiment the chemotherapeutic agent is cytarabine (ARA-C).

In an embodiment the chemotherapeutic agent is quizartinib (AC220).

In an embodiment the chemotherapeutic agent is cytarabine (ARA-C) and the cancer is AML.

In an embodiment the chemotherapeutic agent is quizartinib (AC220) and the cancer is AML.

Specific examples of other anti-cancer agents include, but are not limited to

(i) a vaccine (e.g., IMCgp100, Prophage G-100 & G-200, GV-1001, IMA-950, CV-9201, CV-9104, Ad-RTS-hIL-12, ETBX-011, Cavatak, JX-594, ColoAd1, GL-ONC1, ONCOS-102, CRS-207, ADU-623, Dorgenmeltucel-L, HyperAcute Prostate, FANG vaccine, MGN-1601, HPV vaccine and Tarmogens such as GI-4000);

(ii) anti-cancer reactive mononuclear blood cells (MNBCs);

(iii) a cytokine capable of inducing activation and/or proliferation of a T cell;

(iv) an immune-check point modulator e.g., a PD1 antagonist, PDL-1 antagonist, CTLA-4 antagonist, LAG-3 antagonist, TIM-3 antagonist, KIR antagonist, IDO antagonist, OX40 agonist, CD137 agonist, CD27 agonist, CD40 agonist, GITR agonist, CD28 agonist or ICOS agonist;

More non-limiting examples are provided WO WO2017/009842 and WO2017/009843, each of which is incorporated by reference in its entirety.

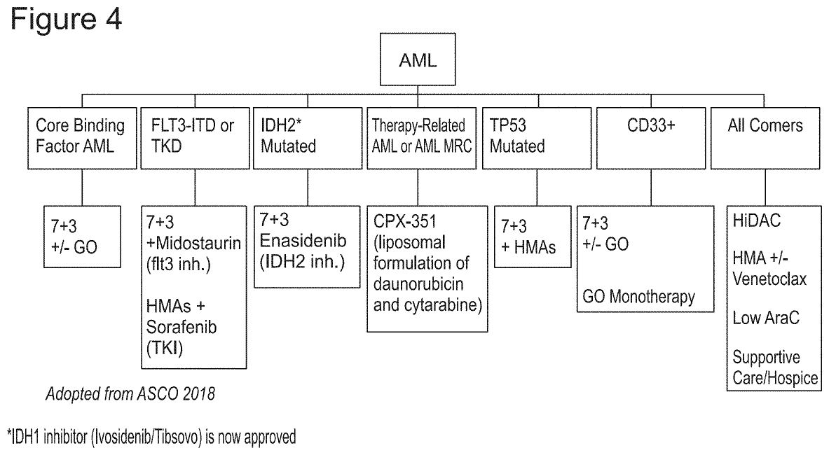

Other treatment modalities that can be used in the treatment of AML include but are not limited to those listed in FIG. 4.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptides and/or the anti-cancer agent accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, intradermal, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The peptide of the invention, the anti-cancer agent or the pharmaceutical composition comprising same can be administered in the same route or in separate routes.

According to a specific embodiment, the peptide of the invention or the pharmaceutical composition comprising same is administered subcutaneously.

According to another specific embodiment, the peptide of the invention or the pharmaceutical composition comprising same is administered intravenously.

According to a specific embodiment, the anti-cancer agent or the pharmaceutical composition comprising same is administered intravenously.

According to a specific embodiment, the anti-cancer agent or the pharmaceutical composition comprising same is administered via a subcutaneous route.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes.

Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Alternative embodiments include depots providing sustained release or prolonged duration of activity of the active ingredient in the subject, as are well known in the art.

Pharmaceutical compositions suitable for use in context of some embodiments of the invention include compositions wherein the active ingredients are contained in an amount effective to achieve the intended purpose. More specifically, according to specific embodiments, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human.

The dosage may vary depending upon the dosage form employed and the route of administration utilized.

The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to specific embodiments the peptide of the invention or the pharmaceutical composition comprising same is administered in a dose ranging between 0.1 to 10 mg/kg of body weight, between 0.1 to 2 mg/kg of body weight, between 0.1 to 1 mg/kg of body weight, between 0.3 to 10 mg/kg of body weight, between 0.3 to 2 mg/kg of body weight, between 0.3 to 1 mg/kg of body weight or between 0.3 to 0.9 mg/kg of body weight.

According to a specific embodiment, the peptide of the invention or the pharmaceutical composition comprising same is administered in a dose ranging between 0.5-2 mg/kg e.g., in a subcutaneous route.

According to another specific embodiment the peptide of the invention or the pharmaceutical composition comprising same is administered at a dose of 0.5-1.5 mg/kg, e.g., in a subcutaneous route.

According to another specific embodiment the peptide of the invention or the pharmaceutical composition comprising same is administered at a dose of 1.25-1.5 mg/kg, e.g., in a subcutaneous route.

For instance, non-limiting examples include:

In r/r AML patients: 2 consecutive days of the peptide (e.g., BL-8040) monotherapy followed by combined administration of the peptide and HiDAC (IV; 1.5 g or 3.0 g/m2/d, based on age) for 5 days and 1-2 cycles

For consolidation therapy in AML patients first remission:
Two or Three cycles (age-based) of consolidation with high-dose Ara-C together with either BL-8040 or Placebo.
Ara-C 1 g/m² per dose for patients older than 60 years and 3 g/m² for patients younger than 60 years. Ara-C is administered IV twice a day (10 am and 10 pm) over 3 hours on day 1, 3 and 5.

For maintenance AML treatment:
BL-8040 (1.25 mg/kg), SC on days 1-3 of each 21-day cycles
Atezolizumab (1200 mg), IV on day 2 of each cycle

The desired dose can be administered at one time or divided into sub-doses, e.g., 2-4 sub-doses and administered over a period of time, e.g., at appropriate intervals through the day or other appropriate schedule.

According to specific embodiments, the peptide of the invention, the anti-cancer agent or the pharmaceutical composition comprising same is administered multiple times e.g. 2-10, over a period of time e.g. for several days to several weeks at appropriate intervals e.g. once a day, twice a week, once a week, once every two weeks, once a month, once every 3 to 6 months.

In the case of a combined treatment e.g., co-treatment of the peptide with a chemotherapeutic agent, they can be administered concomitantly (at about the same time in a single formulation or in separate formulations) or sequentially.

In some embodiments the peptide is administered at least 1 hour, at least 2 hours, at least 4 hours, at least 8 hours, at least 12 hours, at least 1 day, at least 2 days, at least 3 days, at least 4 days, at least 5 days, at least 6 days, at least 1 week, or at least 1 month prior to the administration of the other drug e.g., chemotherapeutic agent.

In some embodiments the peptide and the other drug e.g., chemotherapy, are administered sequentially by within 1 hour, within 2 hours, within 4 hours, within 8 hours, within 12 hours, within 1 day, within 2 days, within 3 days, within 4 days, within 5 days, within 6 days, within 1 week, or within 1 month.

According to some embodiments, the peptide is administered between 1 to 24 hours prior to the administration of the other drug e.g., chemotherapeutic agent. According to some embodiments, the peptide is administered between 1 to 8 hours prior to the administration of the other drug e.g., chemotherapeutic agent.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According an aspect of the present invention there is provided an article of manufacture identified for use in treating cancer, comprising a packaging material packaging a peptide having an amino acid sequence as set forth in SEQ ID NO: 1 or an analog or derivative thereof and an anti-cancer agent as described herein.

The peptide and the agent may be packaged in the same container or in separate containers; each possibility represents a separate embodiment of the present invention.

According to specific embodiments, the peptide and the agent are in separate containers.

According to specific embodiments, the peptide and the agent are in separate formulations.

According to other specific embodiments, the peptide and the agent are in a co-formulation.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N. Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### BL-8040 Mediated CXCR4 Receptor Occupancy and Induced Mobilization and Differentiation of AML Blasts in Relapsed and Refractory AML Patients

A phase 2a, open-label, multicenter, dose escalating study was conducted in subjects with relapsed/refractory AML, defined according to World Health Organization (WHO) criteria, including subjects who failed chemotherapy only and those who failed previous Autologous Stem Cell Transplantation (ASCT)/Allogeneic Stem Cell Transplantation (AlloSCT), provided at least 6 months have passed from transplant.

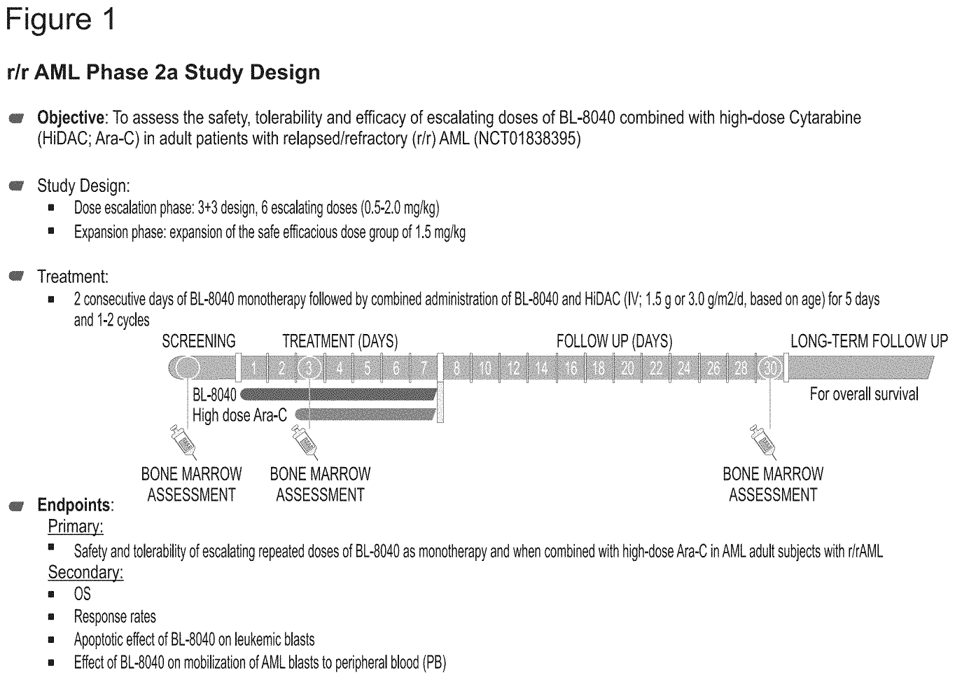

Eligible subjects received subcutaneous (SC) injections of SEQ ID NO: 1 (BL-8040) (“monotherapy period”) over two days (one dose per day that could be administered at one or more injection sites) followed by concurrent administration of BL-8040 with standard salvage chemotherapy (“combination period”) over 5 days (FIG. 1). BL-8040 administration during the “combination period” was also one dose per day at one or more injection sites, at the discretion of the Investigator. During the “combination period,” BL-8040 was administered approximately 4 hours prior to chemotherapy. The chemotherapy consisted of cytarabine (Ara-C) 1.5 or 3 g/m²/d per dose (based on age), administered intravenously (IV) over 3 hours, for 5 days and was not escalated.

Diagnosis and main criteria for inclusion were as follows:

### Inclusion Criteria:

1. Adult men and women subjects aged 18 to 75, inclusive.
2. Confirmed diagnosis of relapsed/refractory AML (WHO criteria).
   Refractory subjects after up to 2 cycles of induction therapy or first complete response (CR1) duration ≤90 days.
   Relapse occurring >90 days and ≤24 months since CR1.
3. AML relapse >6 months since autologous or allogeneic stem cell transplantation, provided they were in first relapse and:
   No active graft-versus-host disease (GVHD>grade 1).
   No treatment with high dose steroids for GVHD (up to 20 mg Prednisolone or equivalent).
   No treatment with immunosuppressive drugs with the exception of low dose cyclosporine and tacrolimus (blood levels of 0.5-0.6 μg/mL).
4. Clinical laboratory values had to be as follows:
   White blood cell (WBC) <30,000/μL.
   Blasts in PB ≤20,000. Treatment with Hydroxyurea was permitted up to 24 hrs prior to BL-8040 administration to achieve blast counts <20,000 prior to enrollment.
   Creatinine <1.3 mg/dL; if Creatinine was >1 mg/dL the Creatinine clearance had to be >40 mL/min as calculated using the Cockcroft-Gault formula.
5. Women of childbearing potential and all men had to agree to use an approved form of contraception (e.g. oral, transdermal patch, implanted contraceptives, intrauterine device, diaphragm, condom, abstinence or surgical sterility) prior to study entry and for the duration of study participation through 30 days following the last dose of BL-8040. Confirmation that female subjects were not pregnant had to be established by a negative serum β-human chorionic gonadotropin (β-hCG) pregnancy test result obtained during screening. Pregnancy testing was not required for post-menopausal or surgically sterilized women.
6. Subject was able and willing to comply with the requirements of the protocol.
7. Subject was able to voluntarily provide written informed consent.

### Exclusion Criteria:

1. Administration of conventional chemotherapy within 2 weeks of enrollment date. In the event that subjects received chemotherapy >2 weeks prior to the date of enrollment, they could be included provided they had recovered from the associated non-hematological toxicities to ≤grade 1.
2. Life expectancy of ≤2 months.
3. Known allergy or hypersensitivity to any of the test compounds, materials or contraindication to test product.
4. Use of investigational device or agents within 2 weeks of enrollment date.
5. Low Performance Status (ECOG >2).
6. O₂ saturation <92% (on room air), evidence of Tumor Lysis Syndrome (TLS) >grade 2 (according to the Cairo-Bishop criteria) or leukostasis.
7. Abnormal liver function tests:
   Serum aspartate transaminase (AST/SGOT) or alanine transaminase (ALT/SGPT) 2× upper limit of normal (ULN).
   Serum bilirubin. Total bilirubin >2.0 mg/dL (34 μmol/L), conjugated bilirubin >0.8 mg/dL.
8. Left ventricular ejection fraction <40%.
9. History of myocardial infarction or cerebrovascular accident within 6 months of enrollment date.
10. Presence of active, uncontrolled infection.
11. Known central nervous system disease (e.g., Alzheimer's disease).
12. Acute promyelocytic leukemia.
13. Exposure to high dose Ara-C within 6 months of enrollment.
14. Subject had a concurrent, uncontrolled medical condition, laboratory abnormality, or psychiatric illness which could place him/her at unacceptable risk, including, but not limited to:
   Subject had been diagnosed or treated for another malignancy within 3 years of enrollment, except in situ malignancy, or low-risk prostate, skin or cervix cancer after curative therapy.
   A co-morbid condition which, in the view of the Investigators, rendered the subject at high risk from treatment complications.
15. Female subjects who were pregnant or breastfeeding.
16. Prior clinically significant grade 3-4 non-hematological toxicity to high dose Ara-C or grade ≥2 of neurological toxicity.
17. Seropositive for HIV antibodies (HIV1 and HIV2), Hepatitis C antibody (Hep C Ab) or a Hepatitis B carrier (positive for Hepatitis B surface antigen [HBsAg]).

The first part of the study (Part 1) included escalating dose groups and was considered the ‘escalation phase’. Six dose levels (see Table 4) were investigated starting at dose level 1. Subjects were accrued in a conventional 3+3 design. BL-8040 at a dose of 1.5 mg/kg was selected for the expansion phase of the study (Part 2).

TABLE 4

| Dose | BL-8040 Dose (free base) | Sample |
|---|---|---|
| Level | Per SC Injection (mg/kg) | Size |
| 1 | 0.5 | 3 |
| 2 | 0.75 | 3 |
| 3 | 1 | 6 |
| 4 | 1.25 | 3 |
| 5 | 1.5 | 3 |
| 6 | 2 | 3 |

Follow-up period started after completion of Ara-C chemotherapy and continued for up to 6 weeks after initiation of salvage chemotherapy with Ara-C, i.e., up to Day 44. Subjects participating in the expansion phase were followed for up to 5 years after completion of the follow-up period. Subjects were contacted by telephone at approximately 3-month intervals (±1 month) after the end of the follow-up period to determine AML status and survival.

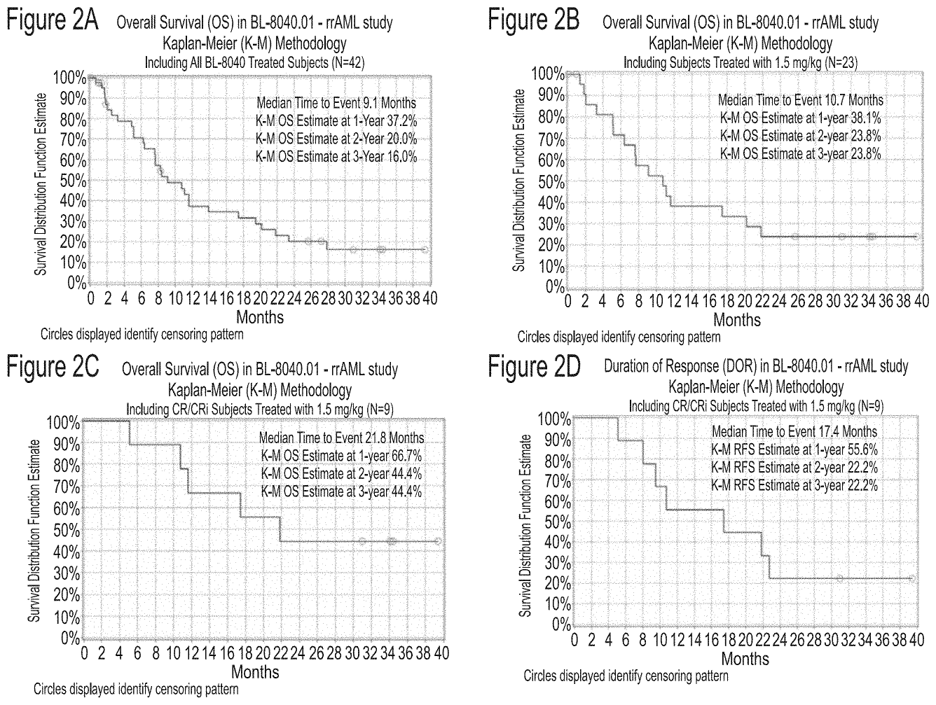

In this Phase 2a study aimed at testing the safety, tolerability and efficacy of escalating doses of BL-8040 combined with high dose Ara-C(HiDAC) in adult patients with relapsed or refractory AML (NCT01838395) the following was demonstrated:
1. BL-8040 is safe and well tolerated at all dose levels (N=42).
2. BL-8040 treatment resulted in composite CR rate (CR and CR with incomplete hematologic recovery, i.e. CRi) of 39% in the expansion group that received 1.5 mg/kg BL-8040 (9/23), composite CR rate of 47% in the refractory patients (9/19) and 13% in relapsed patients (3/23).
3. The median overall survival (mOS) for all patients at all BL-8040 dose levels (N=42) was 9.1 months with 1-year, 2-years and 3-years survival rates of 37.2%, 20% and 16%, respectively (FIG. 2A).
4. In subjects receiving the dose selected for expansion, 1.5 mg/kg (N=23, 54.8%), mOS was 10.7 months with 1-year, 2-year and 3-year survival rates of 38.1%, 23.8% and 23.8%, respectively (FIG. 2B).
5. mOS for responding subjects at the 1.5 mg/kg dose (9/23 with CR/CRi) was 21.8 months, with 1-year, 2-years and 3-years survival rates of 66.7%, 44.4% and 44.4%, respectively (FIG. 2C).
6. mDOR for the responding subjects within the 1.5 mg/kg dose group was 17.4 months and EFS at 1 year, 2 years and 3 years was 55.6%, 22.2% and 22.2%, respectively (FIG. 2D).

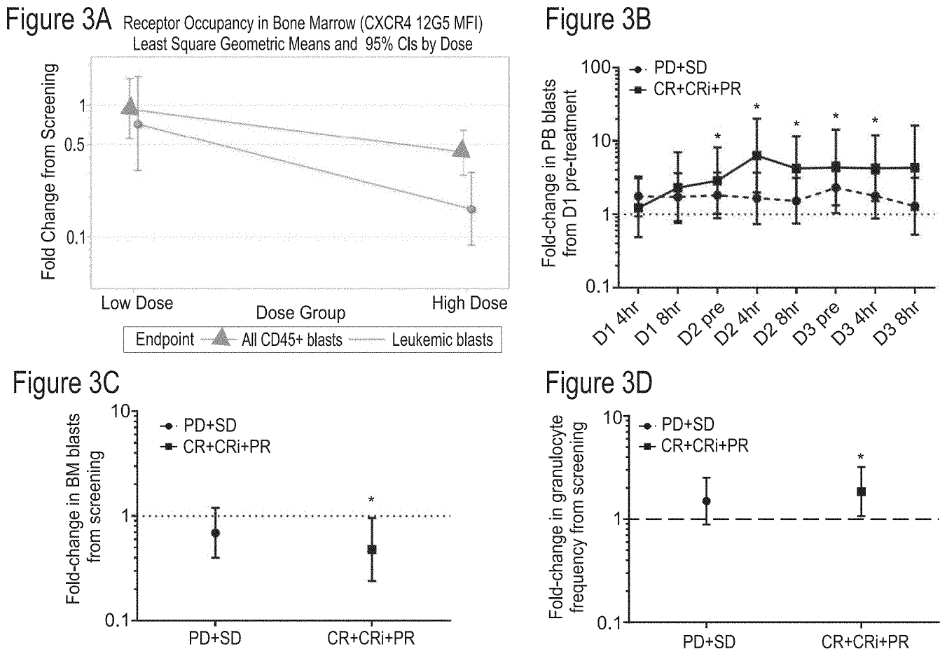

In addition, pharmacodynamic (PD) assessments in this Phase 2a study (NCT01838395) demonstrated the following:
1. High BL-8040 dose levels (1.25, 1.5 and 2 mg/kg) demonstrated CXCR4 receptor occupancy, while low BL-8040 dose levels (0.5, 0.75 and 1 mg/kg) demonstrated insignificant changes in receptor occupancy (FIG. 3A).
2. Correlation between response and mobilization of leukemic blasts: responding patients demonstrated statistically significant increased numbers of mobilized leukemic blasts in peripheral blood following BL-8040 treatment (compared to baseline prior to BL-8040 dosing), compared to patients that did not respond (FIG. 3B). Similar data was seen in the subgroup of patients that received the dose selected for expansion, 1.5 mg/kg (not shown).
3. Responding patients demonstrated decreased numbers of leukemic blasts in BM following BL-8040 treatment (vs baseline prior to BL-8040 dosing), compared to patients that did not respond (FIG. 3C).
4. Statistically significant increased levels of granulocytes were following BL-8040 treatment in BM of responders vs non-responders relative to screening, suggesting that terminal differentiation of leukemic blasts may be an additional anti-leukemic effect of BL-8040 in AML (FIG. 3D).

### Example 2

### Selecting AML Patients for Treatment with BL-8040 Based on CXCR4 Receptor Occupancy

AML blasts from patients (relapsed/refractory, prior to induction, prior to consolidation, or prior to maintenance) are isolated at screening from peripheral blood and/or bone marrow. AML blasts are then treated with various dose levels of BL-8040 or with control and stained for CXCR4 using clone 1D9 (which is able to bind CXCR4 even when the receptor is already bound by BL-8040 and allows determination of total CXCR4 expression) and clone 12G5 (which is unable to bind CXCR4 when the receptor is already bound by BL-8040 and allows determination of receptor occupancy) as well as for the EuroFlow AML panel (CD45, CD34, CD33, CD117, HLA-DR) or the AML MRD panels (Panel 1: CD13, CD15, CD19, CD33, CD34, CD38, CD45, CD71, CD117, HLA-DR; Panel 2: CD4, CD13, CD14, CD16, CD34, CD38, CD45, CD64, CD123, HLA-DR; Panel 3: CD5, CD7, CD11b, CD33, CD34, CD38, CD45, CD56) and analyzed by flow cytometry.

Patients with AML cells that express CXCR4 and demonstrate CXCR4 receptor occupancy of at least 50% by FACS are selected for BL-8040 treatment.

Additional tests to measure CXCR4 expression can be based on assessment of protein expression (by ELISA or other quantitative method), or mRNA level (by RT-QPCR).

Patients with upregulated CXCR4 can also be characterized for AML mutations to enable identification of additional genetic biomarkers to stratify patients. The Illumina TruSight myeloid panel (or other accepted panels) that consists of genes selected by panels of experts in the areas of myeloid hematological cancers to cover key mutations found in AML, or similar methods, can be used.

### Example 3

### Assessment of Minimal Residual Disease (MRD) from Bone Marrow Aspirate (BMA)

AML blasts bearing aberrant marker expression profiles that distinguish them from normal blasts are detected and quantified using multiparametric flow cytometric analysis of bone marrow aspirate (BMA). 1-4 mL of anti-coagulant-treated BMA from the first draw are collected from subjects at screening and possibly at various additional timepoints along the treatment to assure elimination of MRD.

Each BMA sample is incubated with antibody panels against the following cell markers, tested and analyzed by flow cytometry:

Panel 1: CXCR4 (clones 1D9 and 12G5), CD13, CD15, CD19, CD33, CD34, CD38, CD45, CD71, CD117, HLA-DR
Panel 2: CXCR4 (clones 1D9 and 12G5), CD4, CD13, CD14, CD16, CD34, CD38, CD45, CD64, CD123, HLA-DR
Panel 3: CXCR4 (clones 1D9 and 12G5), CD5, CD7, CD11b, CD33, CD34, CD38, CD45, CD56

Patients with AML MRD that express CXCR4 and demonstrate CXCR4 receptor occupancy (at least 50%) by FACS will be selected for BL-8040 treatment.

Correlation between % AML blast cells and clinical response to treatment (prolongation of relapse free survival period) is determined.

### Example 4

### Measurement of CXCR4 and CXCL12 Expression and T Cell Abundance in BM

Bone marrow (BM) biopsies are collected at the clinical sites, fixed in formalin, decalcified to remove any bone contaminants, and then embedded in paraffin. The paraffin blocks are analyzed by immunohistochemistry (IHC) to determine the expression of CXCR4 and CXCL12 and the percentage of AML blasts that are positive for CXCR4 (Abcam ab124824—Clone UMB2 (rabbit)) and CXCL12 (Cell Signaling 97958—Clone D8G6H (rabbit)).

The IHC staining images are reviewed and the number and type of cells in the BM expressing CXCR4 and CXCL12 are enumerated using the markers CD34/CD117 to enumerate total AML blasts and the percentage of AML blasts that express CXCR4 and CXCL12.

Statistical analysis is performed using bivariate correlations to determine the relationship between CXCR4 and CXCL12 expression and clinical response.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of selecting a treatment regimen for a subject diagnosed with a cancer, the method comprising, determining in cancer cells of said subject, CXCR4 occupancy in a presence and an absence of a peptide having an amino acid sequence as set forth in SEQ ID NO: 1 or an analog or derivative thereof, wherein an increase above a predetermined threshold in said CXCR4 occupancy in said presence of said peptide as compared to said absence of said peptide is indicative of suitability of said subject to treatment with said peptide, or analog or derivative.

2. A method of treating a cancer in a subject in need thereof, the method comprising:
(a) administering to the subject a therapeutically effective amount of a peptide having an amino acid sequence as set forth in SEQ ID NO: 1 or an analog or derivative thereof; and
(b) determining an increase above a predetermined threshold in CXCR4 occupancy in cancer cells of the subject following said administering, wherein an increase in CXCR4 occupancy following said administering is indicative of an efficacious treatment.

3. A method of treating a cancer in a subject in need thereof, the method comprising administering to the subject a therapeutically effective amount of a peptide having an amino acid sequence as set forth in SEQ ID NO: 1 or an analog or derivative thereof, wherein said therapeutically effective amount is sufficient to induce at least 50% CXCR4 occupancy in cells of the cancer as can be determined by an assay described in Example 2.

4. (canceled)

5. The method of claim 1, wherein said cancer is dependent on CXCR4 for survival.

6. The method of claim 5, wherein said cancer is a solid tumor.

7. The method of claim 5, wherein said cancer is a hematological malignancy.

8. The method of claim 7, wherein said hematological malignancy is acute myeloid leukemia (AML).

9. The method of claim 1, wherein said cancer cells of the subject are in a biological sample.

10. The method of claim 9, wherein said biological sample is selected from the group consisting of a bone marrow aspirate and a peripheral blood.

11. The method of claim 8, wherein said AML is associated with somatic mutation(s).

12. The method of claim 11, wherein said somatic mutations are in FLT3.

13. The method of claim 1, wherein said receptor occupancy is determined using an antibody which binds peptide-free CXCR4 prior to and post contacting with the peptide.

14. The method of claim 1, wherein said receptor occupancy is determined a first antibody determining total CXCR4 and a second antibody determining peptide-free CXCR4.

15. The method of claim 1, wherein said receptor occupancy is determined by flow cytometry.

16. The method of claim 1, wherein said subject diagnosed with AML is in a stage selected from the group consisting of newly diagnosed prior to induction therapy, prior to consolidation therapy, minimal residual disease prior to maintenance therapy, relapsed stage, refractory stage.

17. The method of claim 1, wherein said treatment or treating is in combination with another treatment modality.

18. The method of claim 17, wherein said another treatment modality is selected from the group consisting of a chemotherapy, targeted therapy and an immune modulator.

19. The method of claim 18, wherein said immune modulator comprises a checkpoint modulator.

20-21. (canceled)

22. The method claim 1, wherein said increase above a predetermined threshold is at least 20%.

23. The method of claim 1, further comprising treating said subject with said peptide, analog or derivative if said suitability is determined.


SEQUENCE LISTING (72 sequences)

SEQ ID NO: 1 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
XRXCYXKXPYRXCR

SEQ ID NO: 2 - protein, 14 aa, Artificial
  SYNTHETIC PEPTIDE
  MOD_RES (1)..(1): N′ ACETYLATION
  MOD_RES (3)..(3): 3-((2-naphthyl) alanine
  MOD_RES (6)..(6): citrulline
  MOD_RES (8)..(8): D-lysine
  MOD_RES (12)..(12): citrulline
RRXCYXKXPYRXCR

SEQ ID NO: 3 - protein, 14 aa, Artificial
  SYNTHETIC PEPTIDE
  MOD_RES (1)..(1): N′ ACETYLATION
  MOD_RES (3)..(3): 3-((2-naphthyl) alanine
  MOD_RES (8)..(8): D-citrulline
  MOD_RES (12)..(12): citrulline
RRXCYRKXPYRXCR

SEQ ID NO: 4 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
RRXCYXKXPYRXCR

SEQ ID NO: 5 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (11)..(12): citrulline
RRXCYXKXPYXXCR

SEQ ID NO: 6 - protein, 14 aa, Artificial
  synthetic peptide
  MOD_RES (1)..(1): N′ acetylated citrulline
  MOD_RES (3)..(3): 3-((2-naphthyl) alanine
  MOD_RES (6)..(6): citrulline
  MOD_RES (8)..(8): D-lysine
  MOD_RES (12)..(12): citrulline
XRXCYXKXPYRXCR

SEQ ID NO: 7 - protein, 14 aa, Artificial
  synthetic peptide
  MOD_RES (1)..(1): N′ acetylated citrulline
  MOD_RES (3)..(3): 3-((2-naphthyl) alanine
  MOD_RES (8)..(8): D-citrulline
  MOD_RES (12)..(12): citrulline
XRXCYRKXPYRXCR

SEQ ID NO: 8 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (11)..(12): citrulline
RRXCYRKXPYXXCR

SEQ ID NO: 9 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-lysine
  MOD_RES (11)..(12): citrulline
XRXCYRKXPYXXCR

SEQ ID NO: 10 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
RRXCYXKXPYRXCR

SEQ ID NO: 11 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (11)..(12): citrulline
  MOD_RES (14)..(14): C′ AMIDATED
RRXCYXKXPYXXCR

SEQ ID NO: 12 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): AMIDATED
XRXCYXKXPYRXCR

SEQ ID NO: 13 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-citrulline
  MOD_RES (12)..(12): citrulline
  MOD_RES (14)..(14): C′ AMIDATED
XRXCYRKXPYRXCR

SEQ ID NO: 14 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (11)..(12): citrulline
  MOD_RES (14)..(14): C′ AMIDATED
RRXCYRKXPYXXCR

SEQ ID NO: 15 - protein, 14 aa, Artificial
  synthetic peptide
  MOD_RES (1)..(1): N′ ACETYLATED citrulline
  MOD_RES (3)..(3): 3-((2-naphthyl) alanine
  MOD_RES (8)..(8): D-lysine
  MOD_RES (11)..(12): citrulline
  MOD_RES (14)..(14): C′ AMIDATED
XRXCYRKXPYXXCR

SEQ ID NO: 16 - protein, 14 aa, Artificial
  synthetic peptide
  MOD_RES (1)..(1): D-glutamic acid
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 17 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
REXCYRKXPYRXCR

SEQ ID NO: 18 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYEKXPYRXCR

SEQ ID NO: 19 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYREXPYRXCR

SEQ ID NO: 20 - protein, 14 aa, Artificial
  synthetic peptide
  MOD_RES (3)..(3): 3-((2-naphthyl) alanine
  MOD_RES (8)..(8): D-glutamic acid
  MOD_RES (12)..(12): citrulline
RRXCYRKXPYRXCR

SEQ ID NO: 21 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYEXCR

SEQ ID NO: 22 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYRXCE

SEQ ID NO: 23 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): C′ amidated
RRXCYXKXPYRXCR

SEQ ID NO: 24 - protein, 14 aa, Artificial
  synthetic peptide
  MOD_RES (3)..(3): 3-((2-naphthyl) alanine
  MOD_RES (6)..(6): D-glutamic acid
  MOD_RES (8)..(8): D-citrulline
  MOD_RES (12)..(12): citrulline
  MOD_RES (14)..(14): c′ amidated
RRXCYXKXPYRXCR

SEQ ID NO: 25 - protein, 14 aa, Artificial
  synthetic peptide
  MOD_RES (3)..(3): 3-((2-naphthyl) alanine
  MOD_RES (6)..(6): D-glutamic acid
  MOD_RES (8)..(8): D-glutamic acid
  MOD_RES (12)..(12): citrulline
  MOD_RES (14)..(14): c′ amidated
RRXCYXKXPYRXCR

SEQ ID NO: 26 - protein, 14 aa, Artificial
  synthetic peptide
  MOD_RES (1)..(1): D-glutamic acid
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 27 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (10)..(10): D-glutamic acid
  MOD_RES (12)..(12): citrulline
  MOD_RES (14)..(14): c′ amidated
RRXCYXKXPXRXCR

SEQ ID NO: 28 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-glutamic acid
  MOD_RES (12)..(12): D-glutamic acid
  MOD_RES (14)..(14): c′ amidated
RRXCYXKXPYRXCR

SEQ ID NO: 29 - protein, 14 aa, Artificial
  synthetic peptide
  MOD_RES (1)..(1): N′ ACETYLATED D-glutamic acid
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 30 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (10)..(10): D-glutamic acid
  MOD_RES (12)..(12): citrulline
  MOD_RES (14)..(14): c′ amidated
RRXCYXKXPXRXCR

SEQ ID NO: 31 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (12)..(12): D-glutamic acid
  MOD_RES (14)..(14): c′ amidated
RRXCYXKXPYRXCR

SEQ ID NO: 32 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
RRXCYXKXPYRXCR

SEQ ID NO: 33 - protein, 14 aa, Artificial
  synthetic peptide
  MOD_RES (1)..(1): Guanyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 34 - protein, 14 aa, Artificial
  synthetic peptide
  MOD_RES (1)..(1): Tetramethylguanyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 35 - protein, 13 aa, Artificial
  synthetic peptide
  MOD_RES (1)..(1): Tetramethylguanyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 36 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): amidated
XRXCYXKXPYRXCR

SEQ ID NO: 37 - protein, 14 aa, Artificial
  synthetic peptide
  MOD_RES (1)..(1): 2-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 38 - protein, 13 aa, Artificial
  synthetic peptide
  MOD_RES (1)..(1): 5-aminopentanoyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 39 - protein, 14 aa, Artificial
  synthetic peptide
  MOD_RES (1)..(1): 2-desamino-arginyl
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 40 - protein, 13 aa, Artificial
  synthetic peptide
  MOD_RES (1)..(1): Guanyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 41 - protein, 13 aa, Artificial
  synthetic peptide
  MOD_RES (1)..(1): Succinyl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 42 - protein, 13 aa, Artificial
  synthetic peptide
  MOD_RES (1)..(1): Glutaryl-arginine
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
XXCYXKXPYRXCR

SEQ ID NO: 43 - protein, 14 aa, Artificial
  synthetic peptide
  MISC_FEATURE (1)..(1): desaminoTMG-APA (formula IV in the specification)
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 44 - protein, 14 aa, Artificial
  synthetic peptide
  MISC_FEATURE (1)..(1): R-CH2 - formula (V) in the specification
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MOD_RES (14)..(14): c′ amidated
XRXCYXKXPYRXCR

SEQ ID NO: 45 - protein, 13 aa, Artificial
  synthetic peptide
  MOD_RES (2)..(2): 3-((2-naphthyl) alanine
  MOD_RES (5)..(5): citrulline
  MOD_RES (7)..(7): D-glutamic acid
  MOD_RES (11)..(11): citrulline
  MOD_RES (13)..(13): c′ amidated
RXCYXKXPYRXCR

SEQ ID NO: 46 - protein, 14 aa, Artificial
  synthetic peptide
  MOD_RES (1)..(1): tetramethylguanyl-arginine
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
XRXCYXKXPYRXCR

SEQ ID NO: 47 - protein, 14 aa, Artificial
  synthetic peptide
  MOD_RES (1)..(1): 6-aminohexanoyl-arginine
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
XRXCYXKXPYRXCR

SEQ ID NO: 48 - protein, 14 aa, Artificial
  synthetic peptide
  MOD_RES (1)..(1): 6-aminohexanoyl-arginine
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 49 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ amidated
RRXCYXRXPYRXCR

SEQ ID NO: 50 - protein, 14 aa, Artificial
  synthetic peptide
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ amidated
RRXCYXRXPYRXCR

SEQ ID NO: 51 - protein, 14 aa, Artificial
  SYNTHETIC PEPTIDE
  MOD_RES (1)..(1): N′ ACETYLATED
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
RRXCYXKXPYRXCR

SEQ ID NO: 52 - protein, 14 aa, Artificial
  synthetic peptide
  MOD_RES (1)..(1): N′ ACETYLATED
  MOD_RES (3)..(3): 3-((2-naphthyl) alanine
  MOD_RES (8)..(8): D-citrulline
  MOD_RES (12)..(12): citrulline
  MOD_RES (14)..(14): C′ AMIDATED
RRXCYRKXPYRXCR

SEQ ID NO: 53 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by a NH-methyl group
XRXCYXKXPYRXCR

SEQ ID NO: 54 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by a NH-ethyl group
XRXCYXKXPYRXCR

SEQ ID NO: 55 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization by NH-isopropyl
XRXCYXKXPYRXCR

SEQ ID NO: 56 - protein, 14 aa, Artificial
  synthetic peptide
  MOD_RES (1)..(1): 4-fluorobenzoyl-arginine
  MOD_RES (3)..(3): 3-((2-naphthyl) alanine
  MOD_RES (6)..(6): citrulline
  MOD_RES (8)..(8): D-glutamic acid
  MOD_RES (12)..(12): citrulline
  MISC_FEATURE (14)..(14): derivatization with a tyramine residue
XRXCYXKXPYRXCR

SEQ ID NO: 57 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
ARXCYRKXPYRXCR

SEQ ID NO: 58 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYAKXPYRXCR

SEQ ID NO: 59 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRAXPYRXCR

SEQ ID NO: 60 - protein, 14 aa, Artificial
  synthetic peptide
  MOD_RES (3)..(3): 3-((2-naphthyl) alanine
  MOD_RES (8)..(8): D-alanine
  MOD_RES (12)..(12): citrulline
RRXCYRKXPYRXCR

SEQ ID NO: 61 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXAYRXCR

SEQ ID NO: 62 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPARXCR

SEQ ID NO: 63 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYAXCR

SEQ ID NO: 64 - protein, 14 aa, Artificial
  synthetic peptide
  MOD_RES (1)..(1): citrulline
  MOD_RES (3)..(3): 3-((2-naphthyl) alanine
  MOD_RES (8)..(8): D-lysine
  MOD_RES (12)..(12): citrulline
XRXCYRKXPYRXCR

SEQ ID NO: 65 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
RRXCYXKXPYRXCR

SEQ ID NO: 66 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-lysine
  MOD_RES (12)..(12): citrulline
  MOD_RES (14)..(14): C′ AMIDATED
RRXCYXKXPYRXCR

SEQ ID NO: 67 - protein, 14 aa, Artificial
  synthetic peptide
  MOD_RES (3)..(3): 3-((2-naphthyl) alanine
  MOD_RES (7)..(7): citrulline
  MOD_RES (8)..(8): D-lysine
  MOD_RES (12)..(12): citrulline
RRXCYRXXPYRXCR

SEQ ID NO: 68 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
  MOD_RES (14)..(14): C′ AMIDATED
RRXCYXKXPYRXCR

SEQ ID NO: 69 - protein, 14 aa, Artificial
  synthetic peptide
  MOD_RES (3)..(3): 3-((2-naphthyl) alanine
  MOD_RES (8)..(8): D-lysine
  MOD_RES (12)..(12): citrulline
RRXCYRKXPYRXCR

SEQ ID NO: 70 - protein, 14 aa, Artificial
  synthetic peptide
  MOD_RES (3)..(3): 3-((2-naphthyl) alanine
  MOD_RES (6)..(6): citrulline
  MOD_RES (8)..(8): D-citrulline
  MOD_RES (12)..(12): citrulline
RRXCYXKXPYRXCR

SEQ ID NO: 71 - protein, 14 aa, Artificial
  synthetic peptide
  MOD_RES (3)..(3): 3-((2-naphthyl) alanine
  MOD_RES (8)..(8): D-citrulline
  MOD_RES (12)..(12): citrulline
RRXCYRKXPYRXCR

SEQ ID NO: 72 - protein, 14 aa, Artificial
  synthetic peptide
  MOD_RES (1)..(1): citrulline
  MOD_RES (3)..(3): 3-((2-naphthyl) alanine
  MOD_RES (8)..(8): D-citrulline
  MOD_RES (12)..(12): citrulline
  MOD_RES (14)..(14): C′ Amidated
XRXCYRKXPYRXCR